(19) United States
(12) Patent Application Publication
(10) Pub. No.: US 2010/0093985 A1
(43) Pub. Date: Apr. 15, 2010

(54) USE OF AT LEAST ONE CYTOSOLIC PHOSPHOLIPASE A2 INHIBITOR AS A MEDICINE FOR SYMPTOMATIC TREATMENT OF MUCOVISCIDOSIS

(75) Inventors: Fariel Dif, Paris (FR); Lhousseine Touqui, Paris (FR)

Correspondence Address: OBLON, SPIVAK, MCCLELLAND MAIER & NEUSTADT, L.L.P., 1940 DUKE STREET, ALEXANDRIA, VA, 22314, US

(73) Assignees: INSTITUT PASTEUR, Paris (FR); INST NAT DE LA SANTE ET DE LA RECHER. MED (INSERM), Paris Cedex 13 (FR)

(21) Appl. No.: 12/295,965

(22) PCT Filed: Apr. 5, 2007
(86) PCT No.: PCT/FR2007/000582

§ 371 Date: Mar. 19, 2009

(30) Foreign Application Priority Data
Apr. 6, 2006 (FR) 0603059

(51) Int. Cl. C07H 21/02; C07C 59/76; C07D 295/02; C07F 9/28; C07D 209/04

(52) U.S. Cl. 536/23.1; 562/577; 548/579; 558/202; 548/469

(57) ABSTRACT

A subject of the invention is the use of at least one cytosolic phospholipase A2 (cPLA2) inhibitor in the preparation of a medicament intended for the preventive and/or curative symptomatic treatment of cystic fibrosis, particularly of the increased secretion of mucus in cystic fibrosis.

The invention is within the field of the symptomatic treatment of cystic fibrosis.

Cystic fibrosis (CF) is an autosomal recessive genetic disease caused by a mutation of the CFTR gene, a gene of 250 kb which codes for a protein of 1480 amino acids called the CFTR (Cystic Fibrosis Transmembrane Conductance Regulator) protein. This protein is part of the family of the chloride channels. To date, more than 1000 mutations responsible for the disease have been described, but the most frequent (discovered in 1989) called ΔF508, is present in approximately 70% of patients. This mutation consists of the deletion of an amino acid, a phenylalanine in position 508, due to a mutation relating to the tenth exon of the gene. The incidence of mutations in the population is approximately 1 in 3000 births, but only homozygotes become ill. Age expectancy is approximately 35 years in developed countries. Heterozygotes or healthy carriers, who are phenotypically normal, represent approximately 4% of the general population.

One of the main consequences of this disease is the modification of the composition of the mucus (the viscosity of which is higher than normal) secreted mainly by the secretory epithelia of the respiratory and digestive mucous membranes. The alteration of the CFTR channel causes changes in the transepithelial ion flux which makes the mucus more viscous and thicker, thus preventing its progression towards the glottis in cystic fibrosis patients. This excess of mucus and absence of mucociliary clearance promotes chronic infection by opportunistic bacteria (such as Pseudomonas aeruginosa, Staphylococcus aureus, Haemophilus influenzae), which can cause an exacerbated inflammatory response in the airways, characterized by a massive inflow of neutrophils. These pathogens are responsible for most episodes of bronchial infection. The lack of control of this inflammatory process, often associated with a bronchial obstruction, causes the destruction of the pulmonary tissues thus causing a progressive loss of respiratory function leading to the death of the patient.

This disease is also characterized by digestive disorders due essentially to a pancreatic exocrine insufficiency present in 90% of patients. These respiratory and digestive symptoms dominate the clinical picture, but respiratory symptoms determine the vital prognosis of patients.

Chronic infection with P. aeruginosa constitutes the main infectious problem and usually marks a watershed in the progression of the disease. Certain so-called “mucoid” strains develop within the mucus in the form of microcolonies encased in an exopolysaccharide matrix.

This mucoid property is virtually specific to the infection. The incidence of mucoid strains increases with age and the progression of the respiratory disease.

Bronchopulmonary symptoms develop in periodic onsets which lead over a few months or several decades to chronic respiratory insufficiency. Complications such as recurrent pneumothorax or hemoptysis can adversely affect the vital prognosis.

As a general rule, death occurs following a worsening of the respiratory symptoms of the progressive infection accompanied by symptoms of right or biventricular heart failure in approximately ⅓ of cases. Currently, cystic fibrosis can only be treated symptomatically. Care is constituted essentially by respiratory kinesitherapy and anti-infection treatment by antibiotic therapy. As well as being burdensome, this treatment must be followed throughout life.

Antibiotic therapy must conform to a certain number of general restrictive principles: appropriate choice of antibiotics, prevention of resistance caused by chronicity of bronchial infection, high doses in treatments of long duration (minimum two weeks), administration by parenteral route only for antipyocyanic antibiotics.

Some molecules allow mucus to be fluidized, in particular rhDNase, a catalytic enzyme for the hydrolysis of extracellular DNA which makes it possible to reduce the viscosity of the mucus which is then easier to remove by kinesitherapy. In the case of chronic progressive respiratory insufficiency, nocturnal oxygen therapy becomes necessary. However, pulmonary transplantation remains a last resort.

Therefore there is always a need for a preventive and/or curative treatment of the symptoms, which would not be as burdensome as current treatments (not to mention transplantation) and which improve the lives of patients.

This is one of the aims of the present invention.

Infection by a pathogen such as Pseudomonas aeruginosa causes the activation of inflammatory proteins such as cytosolic phospholipase A2 (cPLA2). The latter moves towards the plasmic membrane in order to hydrolyze phospholipids and release arachidonic acid (AA).

AA is then metabolized by two distinct enzymatic routes implicated in the inflammation: the cyclooxygenase (COX) route which succeeds in producing prostaglandins, in particular prostaglandin E2 (PGE2); and the lipooxygenase route which leads to the production of leukotrienes, in particular leukotriene B4 (LTB4).

Previous studies have shown the presence of an increased quantity of leukotriene B4 (LTB4) and prostaglandin E2 (PGE2) in bronchoalveolar lavages (BAL) of patients suffering from cystic fibrosis (CF patients). This increase has also been found in an animal model of cystic fibrosis in KO (knockout) mice in which the gene coding for CFTR has been deleted, therefore not producing the CFTR protein (mutant mice called CFTR^{-/-}). Moreover, it has been established that LTB4 and PGE2 are capable of inducing the secretion of mucus.

The inventors have posited the hypothesis of a link between the existence of the mutation of the CFTR gene, causal agent of cystic fibrosis, and the increased secretion of mucus in the lungs, through the increased activity cPLA2.

The inventors then showed that CFTR^{-/-} mice, stimulated by infusion with lipopolysaccharide (LPS) of Pseudomonas aeruginosa which causes inflammation of the airways, secrete more mucus than wild-type mice.

By infusion is meant administration via the airways, more precisely by intratracheal route.

The inventors also showed that this secretion of mucus is inhibited by aspirin, a COX inhibitor.

Similarly, the study by the inventors of the secretion of mucus in the airways of KO mice in which the gene coding for cPLA2 has been deleted (mutant cPLA2^{-/-} mice) shows that the latter is completely absent. Therefore cPLA2 also plays a part in regulating secretion of the mucus in the airways.

Mucus is constituted essentially of mucins (glycoproteins) and MUC5AC is one of the most abundant mucins in the lungs of patients suffering from cystic fibrosis.

In this connection also, the inventors were able to show by immunohistochemical staining that MUC5AC is indeed expressed in CFTR^{-/-} mice which exhibit an increase in the quantity of mucus in the airways.

The inventors have therefore confirmed the hypothesis that they put forward by showing for the first time in CFTR^{-/-} mice a relationship, probably direct, between at least one mutation of the CFTR gene, responsible for cystic fibrosis, and the secretion of mucus, linked to an overactivation of cPLA2.

The level of cPLA2 activity in bronchial homogenates of lung explants of CF patients who had or had not received ATK confirms this possibility. Finally, the inventors were able to show that administration by intraperitoneal route of a specific inhibitor of cPLA2 completely eliminates the secretion of mucus in the airways, a secretion probably caused by infusion of LPS from Pseudomonas aeruginosa in CFTR^{-/-} mice which express cPLA2 at higher levels than CFTR^{+/+} mice.

These results for the first time allow the use of a cPLA2 inhibitor to be envisaged in the symptomatic treatment of cystic fibrosis.

These surprising and unexpected results allow a therapeutic, preventive and/or curative role to be conceived for cPLA2 inhibitors in the treatment of diseases of the airways leading to an increased secretion of mucus, such as for example cystic fibrosis, but also asthma, chronic obstructive lung diseases (COLD) such as for example laryngitis and pharyngitis, pneumonia, influenza, pneumoconiosis, chronic bronchitis and emphysema or also allergic aspergillosis.

Thus a subject of the invention is the use of at least one cytosolic phospholipase A2 inhibitor (cPLA2) in the preparation of a medicament intended for the preventive and/or curative symptomatic treatment of cystic fibrosis, particularly of the increased secretion of mucus by the secretory epithelia of the respiratory and digestive mucous membranes.

By cPLA2 inhibitor is meant any chemical or biological, natural or synthetic molecule, any composition which, whatever the mechanism, causes after administration a reduction, or even a complete inhibition, of the activity of cPLA2 or the expression of the cPLA2 gene. There can be mentioned by way of an example of a cPLA2 inhibitor, ATK (arachidonyl trifluoromethyl ketone; Ackermann et al., J. Biol. Chem., 1995; 270: 445-50), pyrrolidine-1 (Seno et al., J. Med. Chem. 2000; 43: 1041-44), MAFP (methyl arachidonyl fluorophosphonate: Lio et al., Biochim. Biophys. Acta; 1996; 1302: 55-60), ML3196 (Lehr, J. Med. Chem., 1997, 40: 2694-2705), 4-[4-[2-[2-[bis(4-chlorophenyl)methoxy]ethyl-sulphonyl]ethoxy]phenyl]-1,1,1-trifluoro-2-butanone, BMS-229724, (Burke et al., J. Pharmacol. Exp. Ther., 2001, 298: 376-85), 3,3-dimethyl-6-(3-lauroylureido)-7-oxo-4-thia-1-azabicyclo[3,2,0]heptane-2-carboxylic acid or also indoles described in the international application WO 03/048122, as well as the compounds mentioned in patents U.S. Pat. No. 6,630,496 or also U.S. Pat. No. 6,350,892.

According to a particular implementation of the invention, the cPLA2 inhibitor is an siRNA interfering with the expression of the cPLA2 gene.

Preferably, the cPLA2 inhibitor used according to the invention is ATK.

According to another embodiment of the invention, it is possible to use a combination of cPLA2 inhibitors (2 or more) in the preparation of a medicament intended for the treatment of diseases of the airways leading to an increased secretion of mucus, in particular for the symptomatic treatment of cystic fibrosis.

According to the invention, the cPLA2 inhibitor is used in the preparation of a medicament intended for the treatment of cystic fibrosis. It can also be envisaged that the cPLA2 inhibitor be used in the preparation of a medicament intended for the symptomatic treatment of the increased secretion of mucus in asthma, chronic obstructive lung diseases (COLD) such as for example laryngitis and pharyngitis, pneumonia, influenza, pneumoconiosis, chronic bronchitis and emphysema or also allergic aspergillosis.

According to the invention, the cPLA2 inhibitor can be used in the medicament in a quantity comprised between 0.01 mg and 2 g, preferably from 1 mg to 1 g, very preferably from 10 mg to 500 mg.

More particularly, the cPLA2 inhibitor such as for example ATK can in particular be administered in doses comprised between 0.1 mg/kg and 500 mg/kg, preferably 1 mg/kg and 100 mg/kg, very preferably 10 mg/kg and 50 mg/kg.

In the context of the invention, the term “symptomatic treatment” denotes the preventive and/or curative treatment of the symptoms associated with the disease. This treatment also improves the care of patients (reduction in suffering, improvement in life expectancy, slowing of the progression of the disease etc.). Moreover, the treatment can be carried out in combination with other ingredients or treatments, such as in particular other active compounds for treating the pathologies or traumas specified in the present application.

For example it is known that trials involving gene therapy via the airways in cystic fibrosis have produced only average results, well short of the hopes that they had aroused. In the treatment by gene therapy via the airways which involves infusing into the lungs (for example using an aerosol) a nucleic acid-type vector carrying an unmutated and functional CFTR gene so that it reaches the epithelial cells of the surface of the lungs, the vector comes up against the layer of mucus covering said cells. Thus the mucus prevents the vector from reaching its target, at least in a sufficient quantity and this despite prior treatments using mucus fluidizers intended to clear the surface of the pulmonary alveoli of its mucus barrier. All the interest in providing a medicament inhibiting the secretion of mucus which could be administered to complement a gene therapy via the airways is thus understandable. Thus, according to another aspect, the object of the invention is the use of at least one cytosolic phospholipase A2 (cPLA2) inhibitor in the preparation of a medicament intended for the symptomatic treatment of the increased secretion of mucus in cystic fibrosis, said medicament having to be administered to complement a gene therapy for cystic fibrosis.

The medicament according to the invention can take all conceivable administration forms. For example, it can be a liquid such as a syrup or a solution for intramuscular or intravenous injection or a solid such as for example a powder or a tablet.

The administration can be carried out by any method known to a person skilled in the art, preferably by oral route, aerosol route, or by injection, usually by intraperitoneal, intracerebral, intrathecal, intravenous, intraarterial or intramuscular route. Administration by oral route is preferred. In the case of long-term treatment, the preferred administration route is sublingual, oral or transcutaneous.

In general, the daily dose of cPLA2 inhibitor used according to the invention is the minimum dose required to obtain the sought therapeutic effect. This dose will depend on different factors such as the weight of the subject to be treated.

If necessary, the daily dose can be administered in two, three, four, five, six or more doses a day or by multiple sub-doses administered at appropriate intervals during the day.

The chosen quantity will depend on multiple factors, in particular on the administration route, on the duration of administration, on the time of administration, the elimination rate of the compound, the different products used in combination with the compound, the age, weight and physical condition of the patient, as well as his medical history, and all the other information known in medicine.

Moreover, the medicaments according to the invention can comprise at least one other therapeutically active ingredient for use simultaneously, separately or spread over time, in particular during treatment of a subject suffering from a pathology as defined above.

The medicaments according to the invention advantageously include one or more excipients or inert, i.e. pharmaceutically inactive and non-toxic, vehicles. There may be mentioned for example saline, physiological, isotonic, buffered solutions etc. that are compatible with a pharmaceutical usage and known to a person skilled in the art. The medicaments can contain one or more agents or vehicles chosen from dispersants, solubilizers, stabilizers, preservatives, etc. Agents or vehicles that can be used in (liquid and/or injectable and/or solid) formulations are in particular methyl cellulose, hydroxymethyl cellulose, carboxymethyl cellulose, cyclodextrins, polysorbate 80, mannitol, gelatin, lactose, vegetable or animal oils, acacia, etc. The compositions can be formulated in the form of injectable suspensions, gels, oils, tablets, suppositories, powders, gelatin capsules, capsules, etc., optionally by means of pharmaceutical forms or devices ensuring a sustained and/or delayed release. For this type of formulation, an agent such as cellulose, carbonates or starches is advantageously used.

The following examples illustrate the invention without limiting it in any way.

Material and Methods

Animal Models

The studies were carried out on a cystic fibrosis model established in C57BL/6J mice (Cftr^{tmUNC} mice) kept on a mixed gene pool by the “Centre de Distribution, de Typage et d'Archivage Animal” UPS44 CNRS in Orléans. Wild-type CFTR^{+/+} and mutant CFTR^{-/-} mice were weaned at 3-4 weeks and fed with food supplemented with a commercial laxative, Movicol®. Without laxative CFTR^{-/-} mice die rapidly after weaning due to bowel obstructions resulting from a change in the secretion of fluids in the digestive tract.

The experiments were conducted on groups of at least 5 mice aged 5-7 weeks for each category of treatment.

Mutant cPLA2^{-/-} mice and their wild-type cPLA2^{+/+} homologues, from the same gene pool, were obtained from the “Department of Biochemistry and Molecular Biology, Faculty of Medicine, University of Tokyo” (Uozomi et al., Nature, 1997). These mice were aged 6-8 weeks at the time of the experiment.

Products and Reagents Used
xylazine (Rompum, Bayer-France)
ketamine (Imalgène 1000 Merial, Lyons, France)
LPS from P. aeruginosa, serotype 10 (Sigma, St. Louis, Mo.)
EDTA, phenylmethylsulphoxide and dithiothreitol (Sigma, St. Louis, Mo.)
anti-MUC5AC antibody (clone 45 M1, Neomarkers, Fermont, Calif., USA)
anti-mouse IgG antibody (Dako Cytomation Envision System).
aminoethylcarbazole (Sigma, St. Louis, Mo.).

Experimental Protocol

The products used were dissolved in an injectable saline solution (or physiological saline) and injected by intratracheal (i.t.) or intraperitoneal (i.p.) route.

The use protocol was as follows:

1) Aspirin (50 mg/kg, i.p.), 30 minutes before LPS, then each day, once a day;

2) ATK (20 mg/kg, i.p.), 30 minutes before LPS and 24 hours after.

The mice were anesthetized with 2% xylazine (8 mg/kg) and ketamine 1000 (40 mg/kg) before the infusion of LPS (330 μg/kg, i.t.).

Bronchoalveolar lavages (BALs) and lung samplings were carried out 4 days after the infusion of LPS, as indicated below:

Bronchoalveolar Lavages (BAL)

The mice were anesthetized with pentobarbital (i.p.) at 200 mg/kg and their trachea cut and a cannula inserted. The BALs were carried out by successive lavages with 1 ml of injectable saline solution containing 5 mM of EDTA and protease inhibitors (phenylmethylsulphoxide: 5 mM, dithiothreitol 5 mM).

The cells were counted by an automatic counter, Counter ZM (Coultronic, Margency, France). This allows the state of inflammation of lungs to be shown.

The identification of the cell types was carried out by Diff-Quick coloration (Baxter Dade AG, Dudingen, Germany) after cytocentrifugation (Hettich-Universal). This makes it possible to count the proportion of neutrophilic polynuclear cells which reflect inflammation.

The results are expressed in the number of cells per ml of BAL.

Detection of MUC5AC by ELISA

50 μl of each lavage (BAL) sample was incubated for 1 hour with 50 μl of a bicarbonate-carbonate mixture (50/50) at 40° C. in the wells of 96-well plates (Nuc).

After drying, the wells were washed 3 times with PBS then saturated with BSA 2%, fraction V (Sigma) for 1 hour at ambient temperature.

The wells were washed again 3 times with PBS. 50 μl of a primary antibody directed against MU5AC (clone 45 M1) diluted 1/100 in PBS/Tween20 0.05% were then placed in each well and incubation was continued for 1 hour at ambient temperature.

The wells were washed again 3 times with PBS. 100 μl of secondary anti-mouse IgG antibody combined with peroxidase (1: 10,000) was then placed in each well and incubation continued for 1 h at ambient temperature.

After washing the wells 3 times with PBS, the presence of the protein was shown using 3,3′,5,5′-tetramethylbenzidine (TMB) peroxidase, then the reaction was stopped with 1M of H₂SO₄. The absorbance was measured at 450 nm. The results are expressed in milligrams of protein per ml of BAL.

Fixation of the Lungs and Histological Analyses

The lungs were infused with 4% formaldehyde to remove the blood and immersed in 4% formaldehyde for 48 hours at 4° C. before being added to paraffin.

Sections 5 μm thick were stained with hemotoxylin/eosin, periodic acid-Schiff (PAS) and alcian blue (AB; pH 2.4) according to standard methods (Histologie Normale et Pathologique. P. GANTER & G. Jolies, Gauthier-Villars).

After microscopic examination of the sections stained with alcian blue, which colours the mucus, the positive bronchi and cells were counted. The “number of positive bronchi/number of positive cells” ratio makes it possible to establish the score which serves to evaluate the results of the mucus production studies (see below).

Immunohistochemistry

The paraffin sections were treated with the antibody directed against MLJ5AC (clone 45 M1) for 2 hours at ambient temperature, at a concentration of 8 μg/ml in PBS 1×.

These sections were then washed with PBS and incubated for 30 minutes with the anti-mouse IgG antibody, combined with peroxidase.

Visualizations were carried out with aminoethylcarbazole.

Western Blot

The mouse lungs were homogenized with lysis buffer (RLT buffer from Quiagen). The homogenates were centrifuged, and equivalent quantities of proteins (10-50 μg) were deposited on 7.5% electrophoresis gel (tris/glycine/SDS polyacrylamide).

The proteins were then transferred to a nitrocellulose membrane (Millipore) treated beforehand with 5% milk PBS/Tween 1%.

The membrane was then incubated for 1 h at ambient temperature in the presence of the primary antibody, then washed 3 times with the PBS/Tween 1%.

The membrane was then incubated for 1 h at ambient temperature in the presence of the secondary antibody, then washed with PBS/Tween 1%.

The presence of the proteins on the membrane was then detected using the ECL system (Amersham 2106) which produces chemiluminescence with peroxidase, according to the supplier's recommendations. The membrane was then exposed to contact with a radiographic film and the latter developed.

An actin control allows the rate of expression of the MU5AC protein to be standardized. The intensity of the bands is measured with an image analyzer.

Measurement of cPLA2 Activity in Bronchial Homogenates of Lung Explants of CF Patients.

The measurements were carried out according to Kramer et al. (Biochem. J. et al. 248: 779-785 (1987)), with the modifications provided by Hidi et al. (J. Immunol. 151: 5613-5623 (1993)).

The lung homogenates were prepared according to Filgueiras et al. (Lipids 22: 731-735, (1987)).

50-100 μl of lung homogenates were incubated in Tris-HCl buffer containing 1 mM of calcium and 100,000 cpm/ml of the radioactive phosphatidylcholine (1-palmitoyl-214C-arachidonoyl-sn-glycerophosphorylcholine at 52 mCi/mmole, Amersham).

After an incubation of 30 minutes, the reactions were stopped by the addition of a chloroform/methanol solution (1:1).

The lipids were extracted by the Bligh and Dyer method (Can. J. Biochem (1959) 37:911-918), then evaporated to dryness and subjected to thin-layer chromatography (Merck) in the presence of chloroform/methanol/acetic acid/water (65 43, 1, 3) as a migration solution.

The spots corresponding to phosphatidylcholine (PC) were then recovered and their radioactivity measured using standard scintillation liquid. cPLA2 activity was expressed in counts per minute (cpm).

Results:
Production of mucus in the airways of CFTR^{+/+} and CFTR^{-/-} mice on the 4^{th} day with or without stimulation by LPS from P. aeruginosa.

The “control” mice received a dose of physiological saline.

TABLE 1

| | D4 | Average Score |
|---|---|---|
| CFTR^{+/+} | Control | 0 |
| CFTR^{+/+} | LPS | 0.8 |
| CFTR^{-/-} | Control | 2 |
| CFTR^{-/-} | LPS | 5 |

These results show the involvement of the CFTR gene and CFTR protein in the production of mucus by bronchial epithelium cells.
Measurement of the quantity of the MUC5AC protein secreted in the lungs of CF (CFTR−/−) and wild-type (CFTR+/+) mice with or without stimulation by LPS from P. aeruginosa (ELISA)

TABLE 2

| | MUC5AC (mg/ml) |
|---|---|
| Wild mice (CFTR+/+) | 0.30 |
| CF mice (CFTR−/−)* | 0.80 |
| Wild mice (CFTR+/+) + LPS^{#} | 0.70 |
| CF mice (CFTR−/−) + LPS*^{#} | 1.00 |

The CF mice secrete more MUC5AC in the BALs both under basal conditions and after treatment with LPS compared with WT mice.

* P<0.001 CF mice compared with WT;

# P<0.001 LPS compared with the control.
Production of mucus in the airways of CFTR^{+/+} and CFTR^{-/-} mice on the 4^{th} day with or without stimulation with LPS from P. aeruginosa and with administration of aspirin (asp)

The “control” mice received a dose of physiological saline and a dose of aspirin.

TABLE 3

| | D4 | Average Score |
|---|---|---|
| CFTR^{+/+} | Control + asp | 0.25 |
| CFTR^{+/+} | LPS + asp | 0 |
| CFTR^{-/-} | Control + asp | 0.50 |
| CFTR^{-/-} | LPS + asp | 0 |

The scores obtained are to be compared with the corresponding scores of Table 1.

These results show that a COX inhibitor causes an inhibition of the production of mucus by the bronchial epithelium cells, and confirms the involvement of COX in this production.
Production of mucus in the airways of CFTR^{+/+} and CFTR^{-/-} mice on the 4^{th} day with or without stimulation with LPS from P. aeruginosa and administration of ATK.

The “control” mice received a dose of physiological saline and a dose of ATK.

TABLE 4

| | D4 | Average Score |
|---|---|---|
| CFTR^{+/+} | Control + ATK | 0 |
| CFTR^{+/+} | LPS + ATK | 0.50 |
| CFTR^{-/-} | Control + ATK | 0 |
| CFTR^{-/-} | LPS + ATK | 0.17 |

The scores obtained are to be compared with the corresponding scores of Table 1.

These results show that a cPLA2 inhibitor causes an inhibition of the production of mucus by the bronchial epithelium cells.
Production of mucus in the airways of cPLA2^{+/+} and cPLA2^{-/-} mice on the 4^{th} day with or without stimulation with LPS from P. aeruginosa.

The “control” mice received a dose of physiological saline.

TABLE 5

| | D4 | Average Score |
|---|---|---|
| cPLA2^{+/+} | Control | 1.50 |
| cPLA2^{+/+} | LPS | 3.33 |
| cPLA2^{-/-} | Control | 0 |
| cPLA2^{-/-} | LPS | 0 |

These results show the involvement of cPLA2 in the production of mucus by the bronchial epithelium cells.
Measurement of the quantity of MUC5AC messenger RNAs in bronchial homogenates of CFTR+/+ and CTFR−/− mice with or without stimulation with LPS from P. aeruginosa, by Western blot

The primary antibody used is an anti-MUC5AC monoclonal antibody (clone 45 M1, Neomarkers, Fermont, Calif., USA)

The secondary antibody used is an anti-mouse IgG antibody (Dako Cytomation Envision System).

For each sample the measurement of the quantity is related to the measurement of the quantity of actin messenger RNAs measured in the same sample for standardization (MUC5AC mRNA/actin mRNA).

TABLE 6

| | MUC5AC mRNA/actin mRNA |
|---|---|
| CTFR^{+/+} mouse | 0.80 |
| CTFR^{+/+} mouse + LPS* | 0.91 |
| CTFR^{-/-} mouse | 1.20 |
| CTFR^{-/-} mouse + LPS* | 1.37 |

These results confirm the increase of MUC5AC mRNAs in CF mice, whether they are stimulated or not.

* P<0.001 CF compared with WT;
Measurement of the quantity of MUC5AC messenger RNA in bronchial homogenates of CFTR+/+ mice with or without stimulation with LPS from P. aeruginosa and with or without administration of ATK, by Western blot.

Under conditions identical to the previous experiment, the results show that MUC5AC mRNA expression is completely inhibited in non-stimulated CFTR+/+ mice and there is a very small quantity of MUC5AC mRNA in stimulated CFTR+/+ mice.
Measurement of the quantity of cPLA2 messenger RNA in bronchial homogenates of mice.

The primary antibody used is an anti-cPLA2 monoclonal antibody (Cell Signaling or Santa-Cruz).

The secondary antibody used is an anti-mouse antibody (Dako Cytomation Envision System).

For each sample the measurement of the quantity is related to the measurement of the quantity of actin messenger RNA measured in the same sample for standardization (MUC5AC mRNA/actin mRNA).

TABLE 7

| | cPLA2 mRNA/actin mRNA |
|---|---|
| CTFR^{+/+} mouse | 0.44 |
| CTFR^{+/+} mouse + LPS* | 0.80 |
| CTFR^{-/-} mouse | 0.60 |
| CTFR^{-/-} mouse + LPS* | 0.75 |

These results confirm the increase in MUC5AC mRNAs in CF mice, whether they are stimulated or not.

* P<0.001 CF compared with WT.
Measurement of cPLA2 activity in bronchial homogenates of lung explants of CF patients.

TABLE 8

| | cPLA2 activity (cpm) |
|---|---|
| non-CF patients | 420 |
| CF patients | 1830 |
| CF patients + ATK | 506 |

These results show that the use of a cPLA2 inhibitor returns the secretion of mucus in a CF patient to that of a non-CF patient. Histological and immunohistochemical studies.

Histological or immunohistochemical analyses carried out on sections of mouse lungs confirm the results obtained in the measurements of the production of mucus by bronchial epithelium cells.

1. A method of using at least one cytosolic phospholipase A2 (cPLA2) inhibitor in the preparation of a medicament intended for the symptomatic treatment of cystic fibrosis.

2. The method according to claim 1, characterized in that the cPLA2 inhibitor is any natural or synthetic chemical or biological molecule, or any composition which causes after administration a reduction, or even a complete inhibition, of the activity of cPLA2 or the expression of the gene coding for cPLA2.

3. The method according to claim 1, characterized in that the cPLA2 inhibitor is chosen from ATK, pyrrolidine-1, MAFP, ML3196, BMS-229724, 3,3-dimethyl-6-(3-lauroylureido)-7-oxo-4-thia-1-azabicyclo[3,2,0]heptane-2-carboxylic acid, siRNA or also indoles.

4. The method according to claim 3, characterized in that the cPLA2 inhibitor is ATK.

5. The method according to claim 1, characterized in that the cPLA2 inhibitor is used in the medicament in a quantity comprised from 0.01 mg to 2 g, preferably from 1 mg to 1 g, very preferably from 10 mg to 500 mg.

6. The method according to claim 1, in a preventive and/or curative symptomatic treatment.

7. The method according to claim 1, characterized in that the medicament is liquid or solid.

8. The method according to claim 1, characterized in that the medicament is intended to be administered by oral route, by aerosol route or by injection.

9. A method of using at least one cytosolic phospholipase A2 (cPLA2) inhibitor in the preparation of a medicament intended for the symptomatic treatment of the increased secretion of cystic fibrosis mucus, said medicament being intended to be administered to complement a gene therapy for cystic fibrosis.

10. The method according to claim 5, wherein the cPLA2 inhibitor is used in the medicament in a quantity comprising from 1 mg to 1 g.

11. The method according to claim 9, wherein the cPLA2 inhibitor is used in the medicament in a quantity of from 10 mg to 500 mg.

12. The method according to claim 8, wherein the medicament is administered by intraperitoneal, intravenous, intraarterial or intramuscular route.
